(19) United States
(12) Patent Application Publication
(10) Pub. No.: US 2020/0001334 A1
(43) Pub. Date: Jan. 2, 2020

(54) TREATMENT SYSTEM HAVING DEDICATED PRESSURE RESERVOIRS

(71) Applicant: ASP GLOBAL MANUFACTURING GMBH, Schaffhausen (CH)

(72) Inventors: Dang Minh NGO, Fountain Valley, CA (US); Thuan Steven NGUYEN, Irvine, CA (US)

(21) Appl. No.: 16/455,035

(22) Filed: Jun. 27, 2019

Related U.S. Application Data
(60) Provisional application No. 62/690,719, filed on Jun. 27, 2018.
(60) Provisional application No. 62/851,277, filed on May 22, 2019.

(51) Int. Cl. B08B 9/032; A61B 1/12; B08B 5/02; B08B 9/023

(52) U.S. Cl. CPC B08B 9/0328; A61B 1/125; A61B 1/012; B08B 5/02; B08B 9/023; A61B 1/123

(57) ABSTRACT

A fluid dispensing system and treatment system may be used to purge cleaning fluids from lumens of an endoscope. A portion of a fluid chamber can be filled with liquid by providing liquid through an inlet of the fluid chamber. A sealing pressure can be generated with gas in the fluid chamber and additional filling of the fluid chamber through the inlet can be limited. Liquid from the fluid chamber can be dispensed responsive to the sealing pressure. The dispensing can comprise expelling liquid through an outlet of the fluid chamber utilizing gas. The gas may be pressurized in multiple reservoirs, each one being dedicated to a purge line of the treatment system.

### CROSS-REFERENCE TO COPENDING APPLICATION

The present application claims priority under 35 U.S.C. § 119 to U.S. Provisional Patent Application No. 62/690,719, filed Jun. 27, 2018, and U.S. Provisional Patent Application No. 62/851,277, filed May 22, 2019. The entire contents of these applications are incorporated by reference herein in their entirety

### FIELD

The present disclosure relates to a fluid dispensing system and method of use thereof, and a treatment system and method of use thereof. In various examples, the present disclosure relates to treatment processes.

### BACKGROUND

Various medical devices are used in numerous procedures in the medical field. These devices are as varied as the procedures themselves. As such, proper care of these devices is critical for efficiency of application and the proper corresponding treatment of the patient.

After a medical device, such as an endoscope, is used, the medical device is cleaned, disinfected, and/or sterilized in order to prepare the medical device for its next use. The cleaning, disinfecting, and/or sterilizing may include attaching the medical device to a re-processing machine, such as an automated endoscope re-processor (AER), using a connector (a tubing, a fitting, etc.). In order to clean, disinfect, and/or sterilize the medical device, the AER can circulate a liquid through a lumen of the medical device utilizing a liquid pump. After the cleaning, disinfecting, and/or sterilization process is executed, the medical device is ready for another use.

### SUMMARY

In one aspect, a fluid dispensing system comprising a fluid chamber is provided. The fluid chamber comprises an inlet, an outlet, and a gas port. The inlet and outlet are suitable to transport fluid. The inlet comprises a body and a gate. The body comprises an opening suitable to transport liquid into the fluid chamber. The gate is suitable to form a seal with the body responsive to a sealing pressure within the fluid chamber. The seal is suitable to limit the transport of liquid through the opening. The gas port is in fluid communication with the fluid chamber. The gas port comprises a valve comprising a first position and a second position. The first position is suitable to transport gas through the gas port into the fluid chamber to generate the sealing pressure and the second position is suitable to limit transport of gas through the gas port.

In another aspect, a fluid dispensing system comprising a fluid chamber is provided. The fluid chamber comprises an inlet, an outlet, and a gas port. The inlet and outlet are suitable to transport fluid. The inlet comprises a body and a gate. The body comprises an opening suitable to transport liquid into the fluid chamber. The gate is suitable to substantially occlude the opening to limit the transport of liquid through the opening responsive to a sealing pressure within the fluid chamber. The gas port is in fluid communication with the fluid chamber. The gas port comprises a valve comprising a first position and a second position. The first position is suitable to transport gas through the gas port into the fluid chamber to generate the sealing pressure and the second position is suitable to limit transport of gas through the gas port. The outlet is configured to generate a backpressure to limit transport of liquid through the outlet responsive to a filling pressure in the fluid chamber. The filling pressure is less than the sealing pressure. The sealing pressure is suitable to expel at least one of liquid and gas from the fluid chamber through the outlet.

In yet another aspect, a fluid dispensing method is provided. A portion of a fluid chamber is filled with liquid by providing liquid through an inlet of the fluid chamber. A sealing pressure is generated with gas in the fluid chamber and additional filling of the fluid chamber through the inlet is limited. Liquid from the fluid chamber is dispensed responsive to the sealing pressure. The dispensing comprises expelling liquid through an outlet of the fluid chamber utilizing gas.

In a further aspect, a treatment system comprising a fluid dispensing system and a fluid connector is provided. The fluid connector is suitable to provide fluid communication between a fluid dispensing system and a lumen of a device. The fluid dispensing system is suitable to provide liquid and gas to the lumen in a first stage and a second stage. The first stage is configured to pass fluid substantially comprised of liquid through the lumen. The second stage is configured to pass fluid substantially comprised of gas through the lumen. The second stage is suitable to facilitate movement of the first stage.

In another aspect, a treatment system comprising a fluid dispensing system, a treatment chamber, a fluid connector, a first chamber, a first valve, and a second valve is provided. The cleaning chamber is suitable to receive a device comprising a lumen. The fluid connector is suitable to provide fluid communication between a fluid dispensing system and the lumen. The fluid dispensing system is suitable to provide liquid and gas to the lumen in a first stage and a second stage. The first stage is suitable to pass fluid substantially comprised of liquid through the lumen and the second stage is suitable to pass fluid substantially comprised of gas through the lumen. The second stage is suitable to facilitate movement of the first stage. The first chamber comprises a cavity substantially comprised of gas and the first chamber is in fluid communication with the fluid dispensing system. The first valve is in fluid communication with the fluid dispensing system and the first valve is suitable to control fluid communication between the first chamber and the fluid dispensing system. The second valve is in fluid communication with the first chamber and the second valve is suitable to control fluid communication between an air source and the first chamber.

In yet another aspect, a method for treatment of a device comprising a lumen is provided. The lumen of the device is connected in fluid communication with a fluid dispensing system of a treatment system. Liquid and gas are passed through the lumen utilizing the fluid dispensing system in a first stage and a second stage. In the first stage, fluid substantially comprised of liquid is passed through the lumen. In the second stage, fluid substantially comprised of a gas is passed through the lumen. The second stage facilitates the movement of the first stage.

In a further aspect, a fluid dispensing system comprising a fluid chamber is provided. The fluid chamber comprises an inlet, an outlet, and a diverter. The inlet and the outlet are suitable to transport fluid. The diverter is disposed within the fluid chamber and the diverter is suitable to move within the fluid chamber. The diverter comprises a first end, a second end, and a body. The first end is suitable to limit transport of liquid through the inlet. The second end is suitable to limit transport of liquid through the outlet. The body extends from the first end to the second end. The body is suitable to facilitate a relationship between the movement of the first end and the second end.

In a further aspect, the treatment system may comprise a pressure regulator configured to output a pressure, e.g., about 30 psi, a first purge channel configured to be connected to a first endoscope lumen, a second purge channel configured to be connected to a second endoscope lumen, a first reservoir disposed between and connected to the pressure regulator and the first purge channel, and a second reservoir disposed between and connected to the pressure regulator and the second purge channel. The treatment system may also include a first valve disposed between the first reservoir and the first purge channel and a second valve disposed between the second reservoir and the second purge channel. The first valve, the second valve, or both, may comprise a solenoid valve. The system may also comprise a first pressure sensor connected to the first reservoir and a second pressure sensor connected to the second reservoir. Additionally, a third valve may be disposed between and connected to the pressure regulator and the first reservoir. The third valve may also be connected to a second pressure regulator. The third valve may be configured to place one of the pressure regulators in fluid communication with the first reservoir. Further, the third valve may be connected to the second reservoir such that the third valve may also place one of the pressure regulators in fluid communication with the second reservoir.

As such, the treatment system may be used to purge a cleaning liquid from a first lumen and a second lumen of an endoscope. After the first lumen is connected to a first line of the treatment system and the second lumen is connected to a second line of the treatment system the following method, including variations, may be performed. The method may comprise steps of charging a first reservoir on the first line with a gas, charging a second reservoir on the second line with the gas, flowing the gas from the first reservoir through the first lumen to purge the first lumen of the endoscope, and flowing the gas from the second reservoir through the second lumen to purge the second lumen of the endoscope. In variations of this method, the steps of charging the first reservoir and the second reservoir may occur simultaneously. Further, the step of charging the first reservoir may include pressurizing the first reservoir to a pressure between about 25 psi and about 35 psi, e.g., about 30 psi. Additionally, the steps of charging the first reservoir and the second reservoir may each comprise activating a pressure regulator. Additionally, the first line and the second line may be checked for blockages. Finally, the steps of charging the first reservoir, charging the second reservoir, flowing the gas from the first reservoir through the first lumen, and flowing the gas from the second reservoir through the second lumen may be repeated.

It is understood that the inventions described in this specification are not limited to the examples summarized in this Summary. Various other aspects are described and exemplified herein.

### BRIEF DESCRIPTION OF THE DRAWINGS

The features and advantages of the examples, and the manner of attaining them, will become more apparent and the examples will be better understood by reference to the following description taken in conjunction with the accompanying drawings, wherein:

FIG. 1 is perspective view of a non-limiting example of a fluid dispensing system according to the present disclosure;

FIG. 2A is a system diagram of a non-limiting example of a fluid dispensing system comprising a gate in a first gate position according to the present disclosure;

FIG. 2B is a system diagram of the fluid dispensing system of FIG. 2A comprising the gate in a second gate position;

FIG. 3A is cross-sectional front view of a non-limiting example of a fluid dispensing system comprising a diverter according to the present disclosure;

FIG. 3B is a detailed view of the fluid dispensing system of FIG. 3A comprising the diverter in a first diverter position;

FIG. 3C is a detailed view of the fluid dispensing system of FIG. 3A comprising the diverter in a second diverter position;

FIG. 3D is a cross-sectional perspective view of the fluid dispensing system of FIG. 3B;

FIG. 4 is a system diagram of a non-limiting example of a treatment system according to the present disclosure;

FIG. 5 is a system diagram of another non-limiting example of a treatment system according to the present disclosure; and

FIG. 6 is a flow chart of a method for using the treatment system of FIG. 5.

Corresponding reference characters indicate corresponding parts throughout the several views. The exemplifications set out herein illustrate certain embodiments, in one form, and such exemplifications are not to be construed as limiting the scope of the examples in any manner.

### DETAILED DESCRIPTION

Certain exemplary aspects of the present disclosure will now be described to provide an overall understanding of the principles of the structure, function, manufacture, and use of the devices and methods disclosed herein. One or more examples of these aspects are illustrated in the accompanying drawings. Those of ordinary skill in the art will understand that the devices and methods specifically described herein and illustrated in the accompanying drawings are non-limiting exemplary aspects and that the scope of the various examples of the present invention is defined solely by the claims. The features illustrated or described in connection with one exemplary aspect may be combined with the features of other aspects. Such modifications and variations are intended to be included within the scope of the present invention.

Reference throughout the specification to “various examples,” “some examples,” “one example,” or “an example”, or the like, means that a particular feature, structure, or characteristic described in connection with the example is included in at least one example. Thus, appearances of the phrases “in various examples,” “in some examples,” “in one example”, or “in an example”, or the like, in places throughout the specification are not necessarily all referring to the same example. Furthermore, the particular features, structures, or characteristics may be combined in any suitable manner in one or more examples. Thus, the particular features, structures, or characteristics illustrated or described in connection with one example may be combined, in whole or in part, with the features structures, or characteristics of one or more other examples without limitation. Such modifications and variations are intended to be included within the scope of the present examples.

In this specification, unless otherwise indicated, all numerical parameters are to be understood as being prefaced and modified in all instances by the term “about”, in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification such that amending to expressly recite any such sub-ranges would comply with the requirements of 35 U.S.C. § 112 and 35 U.S.C. § 132(a).

The grammatical articles “a”, “an”, and “the”, as used herein, are intended to include “at least one” or “one or more”, unless otherwise indicated, even if “at least one” or “one or more” is expressly used in certain instances. Thus, the articles are used herein to refer to one or more than one (i.e., to “at least one”) of the grammatical objects of the article. Further, the use of a singular noun includes the plural, and the use of a plural noun includes the singular, unless the context of the usage requires otherwise.

As used herein, the term “substantially comprised” means that a component is present in at least 50% by weight. For example, “substantially comprised” can be 50% to 100% by weight such as, for example, at least 60% by weight, at least 70% by weight, at least 80% by weight, at least 90% by weight, at least 95% by weight, or at least 99% by weight.

A treatment process as described herein may be a cleaning process, a disinfecting process, a sterilization process, the like, and combinations thereof. As used herein a “cleaning process” is meant to mean a treatment process employing a cleaning agent that reduces and/or eliminates a debris such as, for example, a dirt, a dust, a particle, an oil, a protein, a carbohydrate, the like, and combinations thereof. A cleaning agent may be a chemical capable of facilitating the cleaning process such as, for example, water, a soap solution, the like, and combinations thereof. In various examples, the cleaning agent can aid in rinsing of an object.

As used herein, a “disinfecting process” is meant to mean a treatment process that substantially reduces a bioburden. A bioburden may be for example, a bacterium, an archaeon, an eukaryote, a virus, and/or other forms of biological agents. As used herein, “substantially reduce” is meant to mean that at least 50% and up to 99.9% of the bioburden has been removed from the object such as, for example, at least 60% and up to 99.9% of the bioburden, at least 70% and up to 99.9% of the bioburden, at least 80% and up to 99.9% of the bioburden, at least 90% and up to 99.9% of the bioburden, at least 95% and up to 99.9% of the bioburden, or at least and up to 99% and up to 99.9% of the bioburden has been removed from the object. A disinfectant may be a chemical capable of disinfection such as, for example, formaldehyde, glutaraldehyde, orth-phthalaldehyde, quaternary ammonium compounds, alcohol, the like, and combinations thereof. In various examples, alcohol can aid in drying of an object.

As used herein, a “sterilization process” is meant to mean a treatment process which reduces and/or eliminates a bioburden that results in a sterilized object being “substantially free” from bioburden. As used herein “substantially free” is meant to mean that the object is at least 99.9% free from bioburden, in some examples at least 99.99% free, in some examples at least 99.999% free, and in other examples at least 99.9999% free from bioburden. The sterilization process may include, for example, heat, a sterilant, irradiation, pressure, and combinations thereof. A sterilant may be a chemical capable of sterilization. A sterilant may be a chemical capable of sterilization, such as, for example, hydrogen peroxide, ethylene oxide, nitrogen oxide, ozone, glutaraldehyde, formaldehyde, peracetic acid, chlorine, iodine, sodium hydroxide, the like, and combinations thereof.

Some medical devices have a lumen which can contain debris after use that should be treated prior to further use of the medical device. Typically, a liquid pump can circulate a liquid through a lumen of a medical device in order to treat the debris. The liquid pump can cavitate and/or otherwise improperly pump liquid through the lumen. Moreover, the liquid provided by the liquid pump may not have enough momentum and/or force to remove the debris. Additionally, in order to dry the lumen, a separate air pump may have to be used in order to pump air through the lumen. However, having two separate pumps can be burdensome and create operational deficiencies.

In order to ensure the medical device can be efficiently and effectively treated, a fluid dispensing system and method as well as a treatment system and method according to the present disclosure are provided.

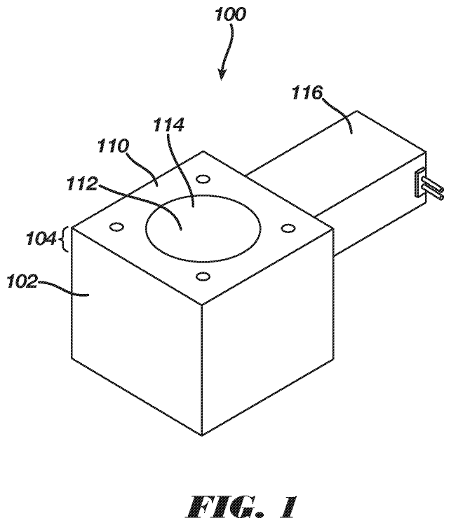

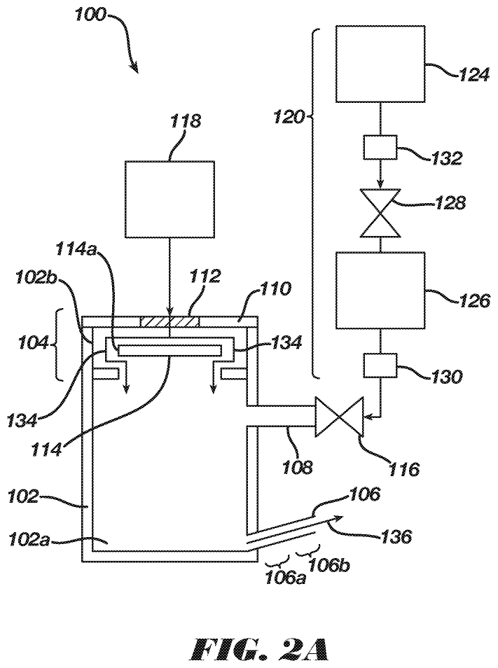

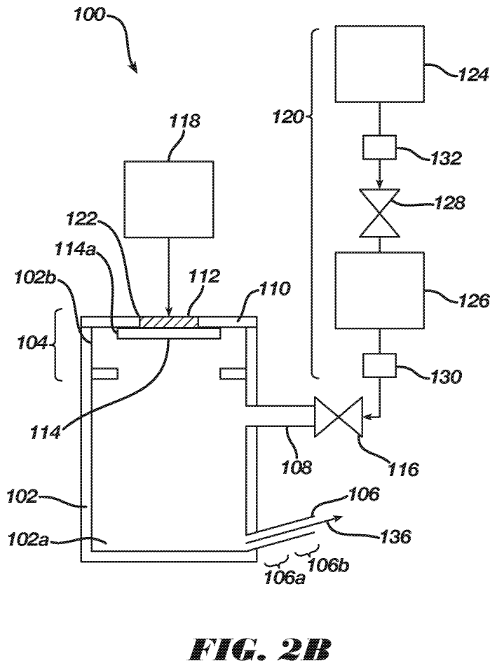

Referring to FIGS. 1 and 2A-B, a fluid dispensing system 100 according the present disclosure is provided. As illustrated, the fluid dispensing system 100 can comprise a fluid chamber 102 comprising a cavity 102a configured to receive a fluid (e.g., liquid and/or gas). For example, the cavity 102a can be in fluid communication with a liquid source 118 via an inlet section, generally, 104 and/or a gas source 120 via a gas port 108. The cavity 102a can be suitable to receive liquid from the liquid source 118 and/or gas from the gas source 120 as illustrated in FIGS. 2A-B. In various examples, the liquid from the liquid source 118 can comprise, substantially comprise, or consist essentially of liquid, and the gas from the gas source 120 can comprise, substantially comprise, or consist essentially of gas.

In various examples, the cavity 102a can be in fluid communication with a plurality of liquid sources including liquid source 118. Each liquid source can comprise the same liquid or a different liquid. For example, each liquid source can comprise at least one of a cleaning agent, a disinfectant, and a sterilant.

In various examples, the cavity 102a can be in fluid communication with a plurality of gas sources including gas source 120. Each gas source can comprise the same gas or a different gas. In various examples, the gas can comprise at least one of air, nitrogen, argon, ethylene oxide, nitrogen oxide, and ozone. In some examples, the gas can substantially comprise air.

The inlet 104 can be disposed between and can be in fluid communication with the cavity 102a and the liquid source 118. The inlet 104 can be suitable to transport fluid into the cavity 102a of the fluid chamber 102. For example, the inlet 104 can comprise a body 110 comprising an opening 112 suitable to receive fluid into the cavity 102a. The liquid from liquid source 118 can flow through the inlet 104 via the opening 112 along a fluid path 134 into the cavity 102a as illustrated in FIG. 2A. In various examples, the fluid dispensing system 100 can be configured in an orientation suitable to gravity fill the cavity 102a of the fluid chamber 102 with liquid through the inlet 104. For example, the liquid source 118 can be positioned at an elevation higher than an elevation of the cavity 102a.

The inlet 104 can comprise a gate 114 which can be suitable to control transport of liquid through the opening 112. As illustrated in FIG. 2A, the gate 114 can be in a first gate position suitable to facilitate transport of liquid into the cavity 102a of the fluid chamber 102 via the fluid path 134. As illustrated in FIG. 2B, the gate 114 can be movable mechanically or by an external force, such as by pressure, into a second gate position suitable to form a seal 122 with the body 110 in order to limit and/or prevent transport of fluid through the opening 112. For example, the gate 114 can substantially occlude the opening 112 such that fluid travel through the opening 112 can be limited and/or prevented when the gate 114 is in the second gate position. In various examples, the body 110 can comprise an elastomer suitable to create the seal 122 with the gate 114. In some examples, the inlet 104 can comprise a check valve (not shown) which can comprise the gate 114 and the body 110.

The position of the gate 114 can be controlled by a pressure within the cavity 102a. For example, responsive to a sealing pressure within the cavity 102a of the fluid chamber 102, the gate 114 can move from the first gate position as illustrated in FIG. 2A to the second gate position as illustrated in FIG. 2B. The sealing pressure can apply a first force to the gate 114 and cause the gate 114 to engage the body 110 of the inlet 104 and form a seal 122 with the inlet 104 such that transport of fluid through the opening 112 in the inlet 104 can be limited and/or prevented. In various examples, the sealing pressure can be greater than an inlet pressure in the inlet 104. In various examples, the sealing pressure is at least 0.1 pounds per square inch (psi) such as, for example, at least 1 psi, at least 2 psi, at least 5 psi, at least 10 psi, or at least 20 psi.

Referring again to FIG. 2A, in the first gate position, the opening 112 can be substantially un-occluded by the gate 114 such that the fluid path 134 can be suitable to transport fluid. The fluid path 134 can be disposed between a wall 102b of the fluid chamber 102 and a perimeter 114a of the gate 114. In various examples, the liquid from liquid source 118 can flow into the cavity 102a via the fluid path 134 of the inlet 104 as illustrated in FIG. 2A. In various examples, the fluid path 134 can be substantially occluded by the gate 114 and transport of liquid through the opening 112 and the fluid path 134 can be limited and/or prevented as illustrated in FIG. 2B.

The pressure within the cavity 102a can be controlled by selectively introducing gas into the cavity 102a utilizing the gas port 108 and/or removing fluid from the cavity 102a utilizing the outlet 106. The gas port 108 can be disposed between and in fluid communication with the cavity 102a of the fluid chamber 102 and the gas source 120. The gas port 108 can comprise a valve 116 suitable to control the flow of gas from the gas source 120 into the cavity 102a. For example, the valve 116 can comprise a first valve position and a second valve position. In the first valve position, the valve 116 can be suitable to transport gas through the gas port 108 into the cavity 102a of the fluid chamber 102. The transport of gas into the gas port 108 can generate the sealing pressure within the cavity 102a. In the second valve position, the valve 116 can be suitable to limit and/or prevent transport of gas through the gas port 108. In various examples, the valve 116 can comprise a solenoid valve. The configuration of the valve 116 is for illustration purposes and other types of valves can be used to control the flow of gas, as known in the art.

In various examples, the gas source 120 can comprise a compressed gas chamber 126 and a gas feed 124. In various examples, the gas feed 124 can comprise at least one of a gas compressor, a gas cylinder, and a gas feed line (e.g., house air). The gas chamber 126 can be disposed between and in fluid communication with the gas port 108 and the gas feed 124.

The gas source 120 can comprise a source valve 128 disposed between and in fluid communication with the compressed gas chamber 126 and the gas feed 124. The source valve 128 can comprise a first source position and a second source position. In the first source position, the source valve 128 can be suitable to transport gas into the compressed gas chamber 126. In the second source position, the source valve 128 can be suitable to limit and/or prevent transport of gas into the gas chamber 126. The compressed gas chamber 126 can be filled with gas utilizing the source valve 128 and gas can be expelled from the gas chamber 126 into the cavity 102a via the gas port 108 utilizing the valve 116. In various examples, the fluid dispensing system 100 can comprise a plurality of source valves including source valve 128. The position of the source valve 128 and the quantity of source valves is for illustration purposes only and there may be a different quantity of source valves and the source valve 128 can be placed in a different position as known in the art.

The gas source 120 can comprise a pressure transducer 130 which can be in fluid communication with the cavity 102a of the fluid chamber 102 and/or the compressed gas chamber 126. The pressure transducer 130 can be configured to determine a pressure in at least one of the gas port 108, the cavity 102a, the compressed gas chamber 126, and the gas feed 124. In various examples, the fluid dispensing system 100 can comprise a plurality of pressure transducers including pressure transducer 130. The position of the pressure transducer 130 and the quantity of pressure transducers is for illustration purposes only and there may be a different quantity of pressure transducers and the pressure transducer 130 can be placed in a different position as known in the art.

The gas source 120 can further comprise a regulator 132 which can be in fluid communication with and disposed between the gas feed 124 and the source valve 128. The regulator 132 can be suitable to control a pressure of gas introduced into the compressed gas chamber 126. For example, the pressure of gas within the gas chamber 126 can be from 0.1 psi to 100 psi such as, for example, 1 to 50 psi, 5 to 40 psi, 10 to 30 psi, or 5 to 10 psi. In various examples, the fluid dispensing system 100 can comprise a plurality of regulators including regulator 132. The position of the regulator 132 and the quantity of regulators is for illustration purposes only and there may be a different quantity of regulators and the regulator 132 can be placed in a different position as known in the art.

The outlet 106 can be suitable to receive fluid from the cavity 102a of the fluid chamber 102 and can be suitable to transport the fluid out of the fluid chamber 102. In various examples, the outlet 106 can be configured to generate backpressure which can limit transport of fluid through the outlet 106 responsive to a filling pressure in the fluid chamber 102. For example, the outlet 106 can be configured to generate backpressure utilizing an inclined outlet fluid path 136. The filling pressure can be less than the backpressure generated in the outlet 106. In various examples, the filling pressure can be less than the sealing pressure. As illustrated in FIGS. 2A-B, fluid path 136 extending through the outlet 106 can have a higher elevation at a second portion 106b of the outlet 106 than at a first portion 106a of the outlet 106. The first portion 106a of the outlet 106 can be disposed adjacent to the cavity 102a. In various examples, the first portion 106a can be disposed between the second portion 106b and the cavity 102a.

The sealing pressure can expel fluid (e.g., at least one of the liquid from the liquid source 118 and the gas from the gas source 120) from the cavity 102a out of the fluid chamber 102 via the outlet 106. In various examples, the fluid chamber 102 can be configured in order to expel fluid in at least two stages responsive to the sealing pressure. The first stage can expel a fluid substantially comprised of liquid and the second stage can expel fluid substantially comprised of gas. In various examples, the second stage can be suitable to facilitate movement of the first stage. For example, the cavity 102a can be filled with liquid prior to generating the sealing pressure in the cavity 102a with the gas from the gas port 108. Upon generating the sealing pressure, the gas from the gas port 108 can push the liquid into the outlet 106 from the cavity 102a. In various examples, the sealing pressure can be greater than the backpressure generated in the outlet 106.

In various examples, the outlet 106 can be in fluid communication with a lumen of a device (not shown) such as, for example, a medical device (e.g., an endoscope). The outlet 106 can transport fluid from the cavity 102a out of the fluid chamber 102 to the lumen of the device responsive to the sealing pressure. The fluid can travel through the lumen of the device and can subject the lumen to a treatment process.

Monitoring pressure utilizing the pressure transducer 130 can indicate the amount of fluid flowing or that has flown through the outlet 106. For example, a pressure in the cavity 102a above a threshold pressure after a time period can indicate that minimal, if any, fluid has been expelled from the outlet 106 and/or to the lumen of the device in fluid communication with the outlet 106 (e.g., there is a blockage in the outlet 106 and/or a blockage in the lumen of the device). The threshold pressure can be equal to or less than the sealing pressure. In various examples, the threshold pressure is greater than the filling pressure.

A pressure in the cavity 102a below the threshold pressure after the time period can indicate that a desired amount of fluid has been expelled from the cavity 102a through the outlet 106 and/or into the lumen of the device (e.g., there is minimal, if any, blockage in the outlet 106 and/or in the lumen of a device).

A method for dispensing a fluid with the fluid dispensing system 100 is further provided and can comprise filling the fluid chamber 102 with liquid by providing liquid through the inlet 104 of the fluid chamber 102. The filling of the fluid chamber 102 can comprise filling at least a portion of the fluid chamber 102 with liquid. In various examples, the gate 114 can be in the first gate position suitable to facilitate filling of the fluid chamber 102. Thereafter, the sealing pressure can be generated in the fluid chamber 102. The sealing pressure can limit and/or prevent additional filling of the fluid chamber 102 through the inlet 104. For example, the gate 114 can move to the second gate position responsive to the sealing pressure and can substantially occlude the opening 112 in the inlet 104. The sealing pressure can be generated by activating the valve 116 to provide gas to the fluid chamber 102. For example, the valve 116 can be suitable to enable transport of gas from the gas source 120 to the fluid chamber 102. In various examples, backpressure can be generated in the outlet 106 of the fluid chamber 102 in order to facilitate filling of the fluid chamber 102. In some examples, filling of the fluid chamber 102 with liquid further comprises gravity filling.

Fluid from the fluid chamber 102 can be dispensed responsive to the sealing pressure. For example, liquid can be expelled through the outlet 106 of the fluid chamber 104 utilizing gas responsive to the sealing pressure. In various examples, gas can be expelled through the outlet 106 of the fluid chamber 104 responsive to the sealing pressure. In various examples, liquid expelled from the fluid chamber 102 can be passed through a lumen of a device (not shown) such as, for example, a medical device (e.g., an endoscope).

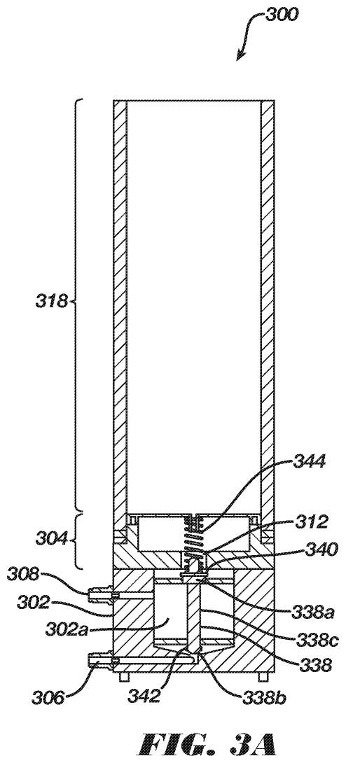

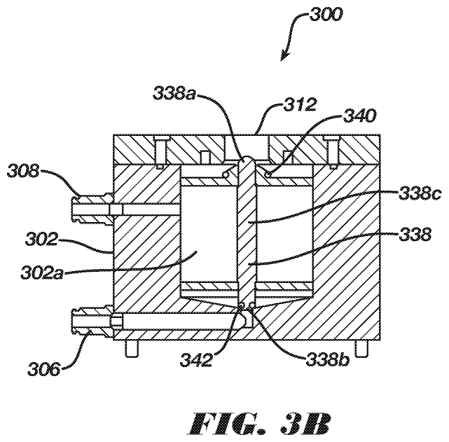

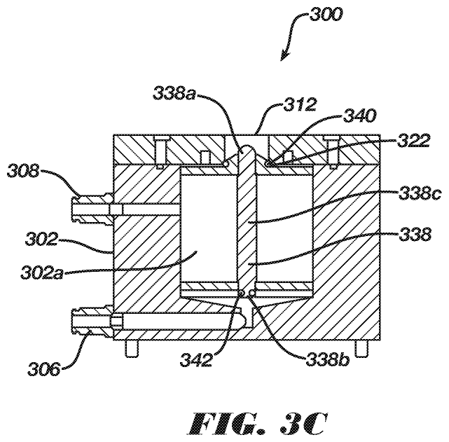

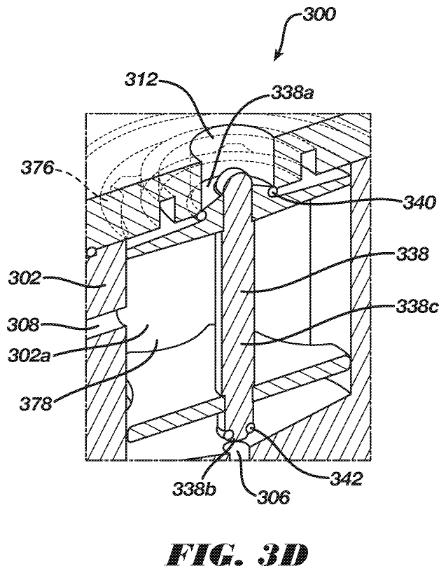

Referring to FIGS. 3A-D, provided is a fluid dispensing system 300 that may be used as a supplement or as a substitute for fluid dispensing system 100, discussed herein. The fluid dispensing system 300, as illustrated, may comprise a diverter 338, a fluid chamber 302, an inlet section, generally, 304, an outlet 306, and a gas port 308. The inlet 304 can be in fluid communication with a liquid source 318 and a cavity 302a of the fluid chamber 302. The inlet 304 can be suitable to transport fluid, such as a liquid, from the liquid source 318 into the cavity 302a of the fluid chamber 302. In various examples, the liquid source 318 can be operatively coupled to the inlet 304 of the fluid chamber 302. The outlet 306 can be in fluid communication with the cavity 302a and can be suitable to transport fluid out of the cavity 302a.

The gas port 308 can be suitable to transport gas into the cavity 302a of the fluid chamber 302 and can control a pressure within the cavity 302a. In various examples, the gas port 308 can comprise valve 116 (FIGS. 1 and 2A-B). For example, the valve 116 can be in fluid communication with the cavity 302a of the fluid chamber 302 and a gas source 120 (FIGS. 2A-B), and in some examples can be configured in the same manner as provided herein with respect to FIGS. 1 and 2A-B.

The diverter 338 can be disposed within the cavity 302a of the fluid chamber 302 and can be suitable to move within the cavity 302a. The diverter 338 can comprise a first end 338a, a second end 338b, and a diverter body 338c. In various examples, the second end 338b can be oppositely disposed from the first end 338a. The diverter body 338c can extend from the first end 338a to the second end 338b and can be suitable to facilitate a relationship between the movement of the first end 338a and the second end 338b. For example, the diverter body 338c can be operatively coupled to the first end 338a and to the second end 338b such that when the first end 338a moves the second end 338b moves.

The first end 338a can be suitable to limit transport of fluid through the opening 312 in the inlet 304. For example, the first end 338a can comprise a sealing member 340 suitable to limit and/or prevent fluid from traversing through the opening 312 such as liquid from liquid source 318. In various examples, the sealing member 340 can comprise at least one of a gasket and a seal. For example, the sealing member 340 can comprise an O-ring.

The second end 338b can be suitable to limit transport of fluid through the outlet 306. For example, the second end 338b can comprise a sealing member 342 suitable to limit and/or prevent fluid from traversing through the outlet 306 such as liquid in the cavity 302a from liquid source 318 and/or gas in the cavity 302a from gas port 308. In various examples, the sealing member 342 can comprise at least one of a gasket and a seal. For example, the sealing member 342 can comprise an O-ring.

The diverter 338 can be configured to move from a first diverter position as illustrated in FIG. 3B to a second diverter position as illustrated in FIG. 3C. In various examples, the diverter 338 can change positions responsive to a sealing pressure within the cavity 302a of the fluid chamber 302. The sealing pressure can apply a first force to the first end 338a of the diverter 338 and cause the first end 338a of the diverter 338 to engage the inlet 304 and form a seal 322 with the inlet 304 such that transport of fluid through the opening 312 in the inlet 304 can be limited and/or prevented. The second end 338b of the diverter 338 can be configured such that force applied to the second end 338b by the sealing pressure can be less than the first force applied to the first end 338a. For example, and as illustrated in FIG. 3D, the second end 338b may have a second fluid opening 376 which is larger than a first fluid opening 378 on the first end 338a.

The first diverter position can facilitate transport of fluid into the cavity 302a of the fluid chamber 302 through the inlet 304 and can be suitable to limit transport of fluid through the outlet 306. For example, in the first diverter position, the second end 338b and sealing member 342 can occlude the outlet 306 and the opening 312 in the inlet may be minimally, if at all, occluded. In various examples, the fluid dispensing system 300 can be orientated such that the cavity 302a can be filled with liquid from the liquid source 318 through the inlet 304 by gravity filling. For example, the fluid dispensing system 300 can be oriented such that liquid source 318 can be positioned at an elevation higher than an elevation of the cavity 302a.

The second diverter position can be suitable to limit transport of fluid into the cavity 302a of the fluid chamber 302 through the inlet 304 and can be suitable to facilitate transport of liquid through the outlet 306. For example, in the second diverter position, the first end 338a and sealing member 340 can occlude the opening 312 in the inlet 304 and the outlet 306 can be minimally, if at all, occluded. In various examples, only one of the opening 312 in the inlet 304 and the outlet 306 can be occluded at a time.

The sealing pressure within the cavity 302a can expel fluid (e.g., at least one of the liquid in the cavity 302a from the liquid source 318 and the gas in the cavity 302a from the gas port 308) from the cavity 302a out of the fluid chamber 302 when the diverter 338 is in the second diverter position. In various examples, the outlet 306 can be in fluid communication with a lumen of a device (not shown) such as, for example, a medical device (e.g., an endoscope). The outlet 306 can transport fluid from the cavity 302a of the fluid chamber 302 to the lumen of the device responsive to the sealing pressure.

In various examples, the fluid dispensing system 300 can comprise a resilient member 344 operatively coupled to the diverter 338. In one example, the resilient member can be a spring-like member. The resilient member 344 can apply a force to the diverter 338 and the force can cause the diverter 338 to change position. For example, the resilient member 344 can move the diverter 338 from the second diverter position as illustrated in FIG. 3C to the first diverter position as illustrated in FIG. 3B. The resilient member 344 can apply a first force to the diverter 338 which can position the diverter 338 in the first diverter position as illustrated in FIG. 3B. The resilient member 344 can maintain the diverter 338 in the first diverter position responsive to a filling pressure in the fluid chamber 302. However, the first force applied by the resilient member 344 on the diverter 338 can be counteracted by a second force applied on the diverter 338 by the sealing pressure within the cavity 302a. When the second force is greater than the first force, the diverter 338 can move from the first diverter position as illustrated in FIG. 3B to the second diverter position as illustrated in FIG. 3C.

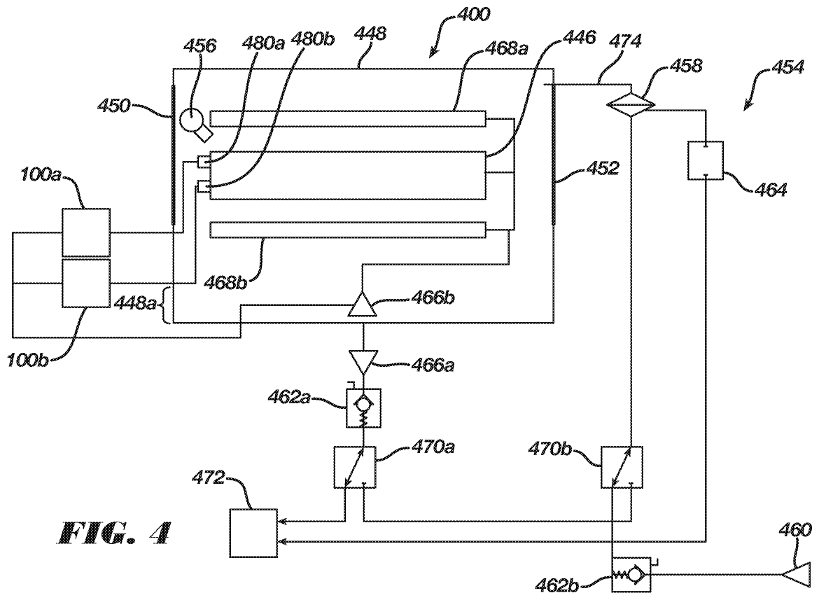

Referring to FIG. 4, a treatment system 400 comprising a fluid dispensing system 100a and a treatment chamber 448 is provided. The treatment chamber 448 can be suitable to receive a device 446. In various examples, the device 446 comprises a medical device (e.g., an endoscope). The fluid dispensing system 100a can be an apparatus or system suitable to provide a fluid flow such as, for example, the fluid dispensing system 100 and/or fluid dispensing system 300, discussed herein. In various examples, the treatment system 400 can comprises two or more fluid dispensing systems 100a-b. The fluid dispensing systems 100a-b can share a common gas source or can have separate gas sources (not shown). In like manner, the fluid dispensing systems 100a-b can share a common liquid source or can have separate liquid sources (not shown). For example, the fluid source of each fluid dispensing system 100a-b can be in fluid communication with a shared reservoir (not shown) such that each fluid dispensing system 100a-b can receive fluid from the shared reservoir.

The fluid dispensing system 100a can be configured in fluid communication with a first lumen of the device 446 utilizing a fluid connector 480a. In various examples, the fluid dispensing system 100b can be in fluid communication with the first lumen utilizing the fluid connector 480a or a second lumen of the device utilizing a fluid connector 480b. In various examples, the first and second lumens of the device 446 are different. In various examples, the fluid connectors 480a, 480b can be at least one of a fitting and a tubing.

Each fluid dispensing system 100a-b can control a fluid flow through their respective outlet. Thus, the fluid flow through the respective lumen of the device 446 in fluid communication with each fluid dispensing system 100a-b can be controlled. Each fluid dispensing system 100a-b can dispense at least one of a cleaning agent, a disinfectant, and a sterilant. In various examples, each fluid dispensing system 100a-b can dispense a different fluid into the first lumen of the device 346. In various examples, the fluid dispensing systems 100a-b can be utilized to dispense the same fluid or different fluids into different lumens of the device 446.

The fluid dispensing systems 100a-b can be suitable to provide fluid to the respective lumen of the device 446 in a first stage and a second stage. In the first stage, the fluid dispensing systems 100a-b can be configured to pass fluid substantially comprising liquid through the respective lumen. In the second stage, the fluid dispensing systems 100a-b can be configured to pass fluid substantially comprising gas through the respective lumen. The second stage can be suitable to facilitate movement of the first stage. For example, the liquid from the first stage can be forced through the respective lumen by the gas of the second stage. The first and second stages can be repeated as necessary to treat the respective lumen and/or remove debris from the respective lumen.

The treatment chamber 448 can comprise a single door or at least two doors. For example, the treatment chamber 448 can comprise a first door 450 and a second door 452. The doors 450, 452 can be suitable to receive the device 446 and can enclose the device 446 within the treatment chamber 448. The first door 450 can receive the device 446 from an environment 454 outside of the treatment chamber 448 and facilitate transport of the device 446 into the treatment chamber 448. In various examples, the first door 450 can be operable to load the device 446 into the treatment chamber 448 when the device 446 is in a first state such as, for example, an unclean state. In some examples, the first door 450 may be used to load but may not be operable to unload the device 446 from the treatment chamber 448. In some examples the first door 450 may not be operable when the device 446 is in a second state such as, for example, a clean state. In various examples, the first door 450 can be secured in a locked state after the device 446 has been loaded and/or when the device 446 is in the second state. In various examples, the door 450 can be operable to unload the device 446 from the treatment chamber 448.

After treating the device 446 in the treatment chamber 448, the device 446 can be in a second state and removed from the treatment chamber 448 utilizing the second door 452 of the treatment chamber 448. The second door 452 can receive the device 446 from the treatment chamber 448 and facilitate transport of the device 446 into the environment 454. In various examples, the second door 452 can be operable to unload the device 446 from the treatment chamber 448 when the device 446 is in the second state. The second door 452 may not be operable to load the device 446 into the treatment chamber 448 and the second door 452 may not be operable when the device 446 is in the first state. In various examples, the second door 452 can be secured in a locked state before the device 446 has been loaded and/or when the device 446 is in the first state. In various examples, the door 452 can be operable to load the device 446 into the treatment chamber 448.

The doors 450, 452 can minimize accidental unloading of the device 446 in an improper state. For example, the doors 450, 452 can prevent unloading of a medical device in a dirty state from the treatment system 400 prior to performing a treatment process. In various examples, after removal of the device 446, the treatment system 400 is ready to treat another device.

The treatment system 400 can comprise a gas dryer 456 disposed within the treatment chamber 448. The gas dryer 456 can be suitable to remove liquid and/or debris from an exterior surface of the device 446 utilizing compressed gas. The gas dryer 456 can be in fluid communication with a gas source which may be the same or different from the gas source used by the fluid dispensing systems 100a-b.

The treatment system 400 can comprise a pump such as, for example pump 466a and pump 466b. Pump 466a can be suitable to re-circulate fluid within the treatment chamber 448 through recirculation line 474 and/or remove fluid from the treatment chamber 448 and provide the fluid to the drain 472. The pump 466b can provide fluid to a spray arm such as, for example, spray arm 468a and spray arm 468b. When present, the spray arms 468a, 468b can deposit (e.g., spray) fluid onto the device 446 such as, for example, the exterior of the device 446. The pump 466b can provide fluid to the fluid dispensing systems 100a-b individually or to a shared reservoir (not shown) that each fluid dispensing system 100a-b can be in fluid communication with. The shared reservoir can provide distribution of the fluid received from the pump 466b to the fluid dispensing systems 100a-b. In various examples, the pump 466b may provide fluid to all of or less than all of the fluid dispensing systems 100a-b. The pump 466b can receive the fluid from a basin 448a of the treatment chamber 448.

In various examples, the treatment system 400 can comprise a check valve such as, for example, check valve 462a and check valve 462b. When present, the check valves 462a, 462b can prevent backflow in the fluid lines.

The treatment system 400 can comprise a 3-way valve such as, for example, 3-way valve 470a and 3-way valve 470b. When present, each 3-way valve 470a-b can be suitable to receive an incoming fluid stream and transport the fluid stream in an outgoing fluid path. For example, 3-way valve 470a can receive a fluid stream from the pump 466a and direct the fluid stream to a drain 472 or to the 3-way valve 470a. The 3-way valve 470b can receive a water stream from water source 460 or the fluid stream from 3-way valve 470a and direct the respective stream to the filter 458. The filter 458 can be suitable to remove debris and/or microorganisms from the respective stream and can output the respective stream into the treatment chamber 448.

Filter 458 can be in fluid communication with a pressure relief valve 464 suitable to relieve an excess of pressure caused by overloading and/or clogging of filter 458. The pressure relief valve 464 can be in a closed state while a pressure at the filter 458 is less than a threshold relief pressure. Upon reaching or exceeding the threshold relief pressure, the pressure relief valve 464 can change to an open state and output a fluid stream from the filter 458 to the drain 472. The location and quantity of the filter 458 is for illustration purposes only, and there may be a plurality of filters in various locations in the treatment system 400.

The treatment system 400 can be used to clean the device 446 comprising the lumen. For example, the device 446 in a first state can be introduced into the treatment chamber 448 utilizing the first door 450. A first lumen of the device 446 can be connected and in fluid communication with the fluid dispensing system 100a and/or the fluid dispensing system 100b of the treatment system 400. A fluid can be passed through the first lumen utilizing the fluid dispensing system 100a-b in at least two stages. For example, the first lumen can be subjected to a first stage comprising passing fluid substantially comprised of liquid through the lumen. The first lumen can be subjected to a second stage comprising passing fluid substantially comprised of a gas through the first lumen. The second stage facilitates the movement of fluid of the first stage. For example, the liquid from the first stage can be forced through the first lumen by gas of the second stage. In certain examples, during the at least two stages of passing fluid through the first lumen, the pressure of gas entering the fluid dispensing apparatus can be monitored. For example, pressure transducer 130 can be utilized to measure the pressure. Measuring the pressure can ensure that fluid is passed through the first lumen or can indicate that the first lumen may contain a blockage.

The device 446 can comprise a plurality of lumens including the first lumen and a second lumen. The second lumen can be connected in fluid communication with the fluid dispensing system 100a and/or the fluid dispensing system 100b of the treatment system 400. In various examples, the fluid dispensing system 100a can pass fluid through the first lumen of the device 446 and the fluid dispensing system 100b can pass fluid through the second lumen of the device. In various examples, the fluid dispensing system 100a or the fluid system 100b can pass fluid through both the first lumen and the second lumen of the device 446.

The treatment process can include passing a plurality of fluids through the lumen of the device 446 in a plurality of phases. For example, a first phase may include passing a first liquid and a gas through the lumen. A second phase may include passing a second liquid and a gas through the lumen, and in various examples, a third phase may include passing a third liquid and a gas through the lumen. The first liquid, the second liquid, and/or the third liquid can comprise at least one of a cleaning agent, a disinfectant, and a sterilant. In some examples, the first liquid comprises a cleaning agent, the second liquid comprises a first disinfectant, and the third liquid comprises a second disinfectant such as, for example, an alcohol. In various examples, the phases can occur in succession such as, for example, the second phase occurs after the first phase, and the third phase occurs after the second phase. In various examples, the phases can occur in various orders such as the starting with the second phase or third phase and ending with the first phase or second phase.

The treatment system 400 can clean an exterior of the device 446 with a liquid stream. For example, the treatment system 400 can utilize the spray arms 468a, 468b to spray a liquid onto the device 446. The liquid can be re-circulated from the basin 448a of the treatment chamber 448 and/or from the fluid dispensing systems 100a-b.

The treatment system 400 can substantially remove liquid and/or debris from an exterior of the device 446 utilizing compressed gas. For example, the treatment system 400 can utilize the gas dryer 456 to flow compressed air over a surface of the device 446.

The fluid dispensing system and method and the treatment system and method according to the present disclosure can pass a liquid and a gas through the lumen of a device which can efficiently and effectively treat a device, such as a medical device. The fluid dispensing system and method and treatment system and method according to the present disclosure can create a fluid stream with an increased momentum which can treat a lumen of a device effectively and rapidly.

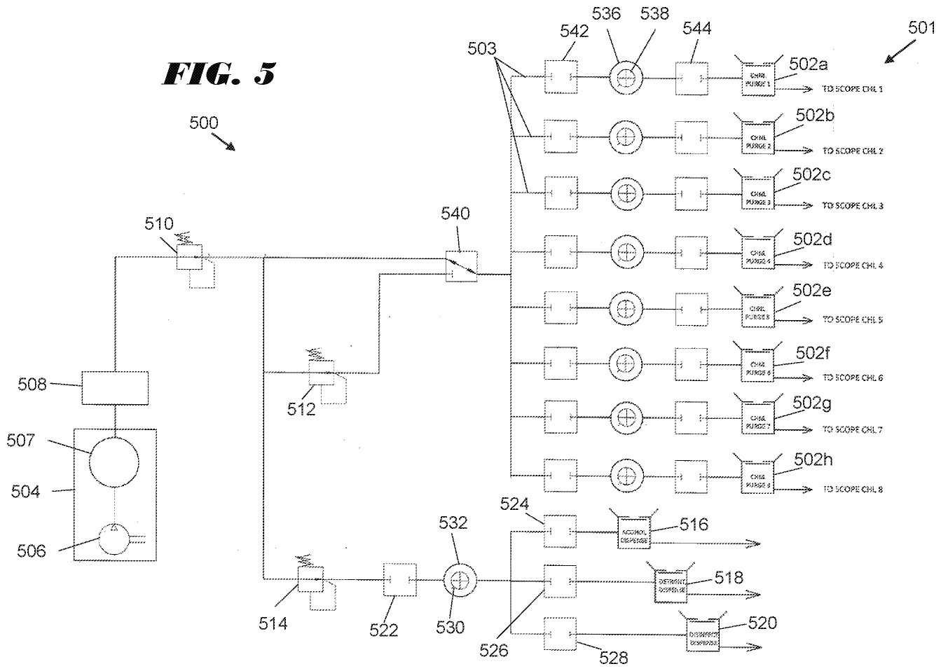

An embodiment of a treatment system 500 suitable for treating an endoscope having a plurality of lumens is shown in FIG. 5. System 500 includes a purge assembly 501 having the same number of, or at least as many, purge channels as the endoscope has lumens. As reflected in FIG. 5, purge assembly 501 includes eight purge channels 502 a-h. Thus, system 500 may be used for disinfecting an endoscope having from one to eight lumens, e.g., two lumens.

System 500 includes a first pressure regulator 504 comprising a gas (e.g., air, nitrogen) compressor 506 that is capable of generating an operating pressure of at least 60 psi. Pressure regulator 504 may additionally include a reservoir 507 in which gas may be collected and stored at the operating pressure. At the outlet of pressure regulator 504, a filter 508, e.g., a biological activated carbon filter, may be provided. A second pressure regulator 510 may be utilized to further adjust the pressure of the gas to be delivered through purge channels 502 to the lumens of the endoscope during a purge cycle in which a cleaning liquid in the endoscope lumens is forcefully ejected therefrom. Inasmuch as endoscopes typically can withstand pressures of up to approximately 35 psi, pressure regulator 510 is capable of lowering the operating pressure to between about 25 psi and about 35 psi, e.g., about 30 psi. A third pressure regulator 512 may optionally be included that may further lower the operating pressure to between about 5 psi and 10 psi, e.g., 8 psi, which may be used to gently check channels 502 and the endoscope lumens for blockages before a higher-pressure purge cycle is commenced. A fourth pressure regulator 514 may be utilized to assist in introducing cleaning liquids from system 500 into the endoscope lumens. Fourth pressure regulator 514 may be in fluid communication with various sources of cleaning liquids, such as an alcohol source 516, a detergent source 518, and a disinfection source 520. Valves, e.g., solenoid valves, 522, 524, 526, and 528, as well as pressure sensor 530 and reservoir 532 may be positioned as reflected in FIG. 5 between fourth pressure regulator 514 and sources 516, 518, and 520 of the cleaning liquids to regulate and monitor the flow of the cleaning liquids through system 500 and ultimately to a medical device, such as an endoscope, connected thereto. The cleaning liquid may flow through features of system 500 such as directly to channel purge assembly 501, or indirectly thereto via the system's treatment or wash chamber 448, described above.

System 500 additionally includes valve 540 between second pressure regulator 510 and third pressure regulator 512 on one side and purge assembly 501 on another side. Valve 540 may be used to control which of the second regulator 510 and third regulator 512 is being used to determine gas pressures downstream thereof.

In addition to purge channels 502 a-h, purge assembly 501 comprises a plurality of fluid lines 503, one corresponding to each of purge channels 502 a-h, through which fluid flows from valve 540 to a corresponding purge channel, e.g., 502a. Each line 503 includes a first line valve 542 and a second line valve 544. Between first line valve 542 and second line valve 544 a reservoir 536 connected to a pressure sensor 538 is disposed. As such a single reservoir 536 on each purge line 503 is dedicated to pressurizing a corresponding purge channel and corresponding endoscope lumen connected thereto.

The foregoing configuration enables improved treatment of endoscope lumens due to shear stresses generated during liquid purging of an endoscope connected to system 500. In particular, this advantage is realized from providing a dedicated pressure sensor 538 and dedicated reservoir 536 on each purge line 503, upstream of purge channels 502 a-h. Each reservoir 536 may be pressurized to a pressure about equal to the maximum pressure that each endoscope channel can withstand, e.g., about 30 psi. After a reservoir has reached that pressure, i.e., after the reservoir has been charged, the corresponding valve 544 may be opened to release the pressurized gas through the corresponding purge channel 502 and through the corresponding endoscope channels to purge the channels of any liquid therein. This configuration allows for the pressure in channels 502a-h and the corresponding endoscope lumens to increase rapidly or immediately from a lower pressure, e.g., ambient pressure, to the pressure in reservoir 536, which the inventors have found creates greater shear stresses in the cleaning liquid against the walls of the endoscope's lumens than simply activating a pressure regulator upstream, such as second pressure regulator 510. Moreover, this configuration enables time savings because each of the reservoirs 536 on each purge line 503 may be pressurized concurrently such that purging following pressurization may also be performed concurrently, thereby reducing the likelihood that a pressure drop may result from using a single pressure regulator to pressurize each line concurrently. Further, dedicating a reservoir for each purge line 503 eliminates the possibility that pressurized gas would favor flowing through those channels 502a-h connected to larger-diameter endoscope lumens (i.e., paths of least resistance) such that smaller-diameter lumens might not be subject to sufficient flow for generating the increased shear stresses desired to achieve the improved purging described herein.

System 500 may additionally include a processor configured to operate system 500 in an automated manner. Specifically, the processor should be connected to at least each of the valves of system 500, i.e., valves 522, 530, 540, 524, 526, 528, 542, and 544, as well as the pressure sensors of system 500, i.e., sensors 530 and 536. As such, the processor can be configured to receive pressure data from the sensors, and to open and close the valves upon determining that a desired pressure is to be achieved or has been achieved in each of the corresponding gas reservoirs, i.e., chambers 532 and 538, as explained below.

System 500 may thus be used to conduct an improved method for purging an endoscope having lumens filled with a cleaning liquid, such as an alcohol, detergent, or disinfectant. Although system 500 may perform additional methods, such as delivering the cleaning liquids to the endoscope, the focus of the method presented at this juncture is to purge the endoscope of these liquids.

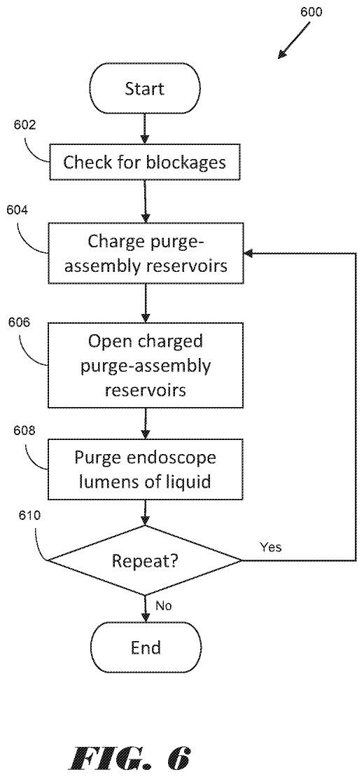

An exemplary method 600 for purging an endoscope is shown in FIG. 6. As described herein as an example, method 600 may be applied to treatment system 500. However, method 600 may be applied to treatment systems having different designs without departing from the scope of this subject matter, including the advantages that may be achieved by providing a dedicated gas reservoir that may be used to purge an individual endoscope lumen. Method 600 may begin at step 602, which comprises checking purge assembly 501 and lumens of an endoscope connected thereto for any blockages. For example, as applied to the treatment system of FIG. 5, the processor may check that valve 522 is closed such that any pressure generated upstream thereof does not cause gas flow downstream thereof. If the processor determines that valve 522 is open, the processor sends a signal to valve 522 to close it. Then, the processor confirms that valve 540 is configured to allow third regulator 512 to be in fluid communication with purge assembly 501 and that second regulator 510 is not such that purge assembly 501 does not receive a gas pressurized to the “purge pressure” from second regulator 510. If the processor determines that this is not the state of the valve, the processor sends a signal to valve 540 to change to this state. Next, the processor opens each of valves of purge assembly 501, i.e., valves 542 and 544 on each purge line 503. As such, pressures generated within first regulator 504 may be regulated down to e.g., about 8 psi, by third regulator 512 such that gas can flow throughout purge assembly 501 and the endoscope connected thereto. Thus, the processor may be configured to monitor pressures at the outlet of each purge line, or alternatively at the outlet of each endoscope, to confirm that the outlet pressure corresponds to a predetermined pressure indicative of no blockage for a given pressure at third regulator 512. Thus, for example, additional pressure sensors may be placed at the outputs of at least one up to each purge line or the outputs of at least one up to each endoscope lumen.

At step 604, the purge assembly is prepared for purging an endoscope connected thereto. For example, as applied to system 500, from at least one to all of the reservoirs 536 are charged, i.e., pressurized to the “purge pressure” and then maintained at that pressure. The processor closes all of the valves 542 and 544, and switches valve 540 to allow fluid communication from second regulator 510 to purge assembly 501, cutting off fluid communication from third regulator 512. Then, from at least one up to each of valves 542 may be opened by the processor, simultaneously or sequentially, allowing fluid communication between second regulator 510 and those reservoirs 536 that have their upstream valve 542 in an open state. As such, these reservoirs 536 become pressurized to the same pressure that the second regulator 510 is configured to output, e.g., about the maximum pressure of the endoscope, e.g., about thirty psi. The processor monitors the pressures in each reservoir 536 via sensors 538 to determine when this pressure, i.e., the “purge pressure,” has been achieved. Upon determining that the purge pressure has been achieved for a given reservoir 536, the processor causes the corresponding valve 542 to close. As such, this given reservoir 536 is in a charged state, and may be considered a “charged reservoir.”

At step 606, the charged purge-assembly reservoirs are open. As applied to system 500, the processor causes valves 544 downstream of a charged reservoir 536 to open. In some variations, this occurs after all reservoirs 536 have been charged. In other variations, this occurs after less than all of the reservoirs 536 have been charged. For example, where the endoscope connected to system 500 includes less than eight lumens, e.g., four lumens, only those four reservoirs 536 of purge channels connected to the endoscope's lumens are charged and have the corresponding valve 544 opened. Additionally, or alternatively, two or more valves 544 may be open simultaneously. Thus, for example, two or more valves 544 may be open after their corresponding reservoirs 536 have been charged. Additionally, or alternatively, two or more valves 544 may be open sequentially. For example, valves 544 may be open after each of the corresponding reservoirs 536 have been charged. Further for example, each valve 544 may be open promptly after charging a corresponding reservoir 536.

In any of these variations, at step 608, gas from the charged reservoirs flows through the lumens of the endoscope, purging them of liquid contained therein. As applied to system 500, gas from charged reservoir(s) 536 advances through any corresponding purge channels 502a-h and endoscope lumens connected thereto.

At step 610, a determination to repeat at least steps 606, 606, and 608, and optionally also step 602, may be made by the processor or an operator of a treatment system. Method 600 may be repeated with or without step 602 whenever a liquid is introduced or reintroduced. For example, these steps may be conducted a first time to purge a first cleaning liquid, e.g., a detergent, a second time to purge a second cleaning liquid, e.g., a disinfectant, and a third time to purge a third cleaning liquid, e.g., alcohol. Additionally, these steps may be repeated multiple times to purge a single cleaning fluid to improve treatment of the endoscope by providing repeated exposure of the lumens to the purge pressure from the dedicated purge reservoirs and the concomitant increased shear stresses along the lumen walls.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

Although various examples have been described herein, many modifications, variations, substitutions, changes, and equivalents to those examples may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed examples. The following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Various aspects of the subject matter according to the present disclosure include, but are not limited to, the aspects listed in the following numbered clauses.
1. A fluid dispensing system comprising:
   a fluid chamber comprising an inlet and an outlet, the inlet and outlet suitable to transport fluid;
   the inlet comprising a body and a gate, the body comprising an opening suitable to transport liquid into the fluid chamber, the gate suitable to form a seal with the body responsive to a sealing pressure within the fluid chamber, the seal suitable to limit the transport of liquid through the opening; and
   a gas port in fluid communication with the fluid chamber and comprising a valve, the valve comprising a first position suitable to transport gas through the gas port into the fluid chamber to generate the sealing pressure, and a second position suitable to limit transport of gas through the gas port.
2. The system of clause 1, wherein the sealing pressure is suitable to expel at least one of liquid and gas from the fluid chamber through the outlet.
3. The system of clauses 1-2, wherein the outlet is configured to generate backpressure to limit transport of liquid through the outlet responsive to a filling pressure in the fluid chamber, the filling pressure is less than the sealing pressure.
4. The system of clause 3, wherein the outlet is configured to generate backpressure utilizing an inclined outlet fluid path.
5. The system of clauses 1-4, wherein the valve comprises a solenoid valve.
6. The system of clauses 1-5, wherein the system is configurable to an orientation suitable to gravity fill the fluid chamber with liquid through the inlet.
7. The system of clauses 1-6, wherein the opening is substantially occluded responsive to the sealing pressure within the fluid chamber.
8. The system of clauses 1-7, further comprising a fluid path disposed between a wall of the fluid chamber and a perimeter of the gate, wherein the fluid path is suitable to transport fluid.
9. The system of clauses 1-8, further comprising:
   a compressed gas chamber comprising a gas inlet and a gas outlet;
   the gas inlet comprising a source valve, the source valve comprising a first position suitable to transport gas into the gas chamber and a second position suitable to limit transport of gas into the gas chamber; and
   the first position of the valve of the gas port suitable to transport gas into the gas port from the gas chamber and the second position of the valve of the gas port suitable to limit transport of gas into the gas port from the gas chamber.
10. The system of clauses 1-9, wherein the gate is configured to move from a first gate position to a second gate position responsive to the sealing pressure within the fluid chamber, the first gate position suitable to facilitate transport of liquid into the fluid chamber, and the second gate position suitable to form the seal with the body.
11. The system of clauses 1-10, wherein the body comprises an elastomer.
12. The system of clauses 1-11, wherein the outlet is in fluid communication with a lumen of an endoscope.
13. A fluid dispensing system comprising:
   a fluid chamber comprising an inlet and an outlet, the inlet and outlet suitable to transport fluid;
   the inlet comprising a body and a gate, the body comprising an opening suitable to transport liquid into the fluid chamber, the gate suitable to substantially occlude the opening to limit the transport of liquid through the opening responsive to a sealing pressure within the fluid chamber;
   a gas port in fluid communication with the fluid chamber and comprising a valve, the valve comprising a first position suitable to transport gas through the gas port into the fluid chamber to generate the sealing pressure, and a second position suitable to limit transport of gas through the gas port;
   the outlet is configured to generate a backpressure to limit transport of liquid through the outlet responsive to a filling pressure in the fluid chamber, the filling pressure is less than the sealing pressure; and
   the sealing pressure is suitable to expel at least one of liquid and gas from the fluid chamber through the outlet.
14. A fluid dispensing method comprising:
   filling a portion of a fluid chamber with liquid by providing liquid through an inlet of the fluid chamber;
   generating a sealing pressure with gas in the fluid chamber and limiting additional filling of the fluid chamber through the inlet; and
   dispensing liquid from the fluid chamber responsive to the sealing pressure, the dispensing comprises expelling liquid through an outlet of the fluid chamber utilizing gas.
15. The method of clause 14, further comprising generating backpressure in the outlet of the fluid chamber in order to facilitate filling of the fluid chamber.
16. The method of clauses 14-15, wherein the filling of the portion of the fluid chamber with liquid further comprises gravity filling.
17. The method of clauses 14-16, wherein generating the sealing pressure further comprises activating a solenoid valve to provide gas to the fluid chamber.
18. The method of clauses 14-17, wherein the inlet comprises a gate and limiting additional filling of the fluid chamber further comprises changing the configuration of the gate utilizing the sealing pressure.
19. The method of clauses 14-18, further comprising expelling gas through an outlet of the fluid chamber.
20. The method of clauses 14-19, wherein dispensing liquid from the fluid chamber further comprises passing liquid through a lumen of an endoscope.
21. A treatment system comprising the fluid dispensing system of any one of clauses 1-20.
22. A treatment system comprising:
   a fluid connector suitable to provide fluid communication between a fluid dispensing system and a lumen of a device; and
   a fluid dispensing system suitable to provide liquid and gas to the lumen in a first stage and a second stage, the first stage configured to pass fluid substantially comprised of liquid through the lumen and the second stage configured to pass fluid substantially comprised of gas through the lumen, and the second stage is suitable to facilitate movement of the first stage.
23. The system of clause 22, further comprising a first chamber comprising a cavity substantially comprised of gas, the first chamber in fluid communication with the fluid dispensing system.
24. The system of clause 23, further comprising a pressure transducer in fluid communication with the first chamber.
25. The system of clause 23-24, further comprising:
   a first valve in fluid communication with the fluid dispensing system, the first valve suitable to control fluid communication between the first chamber and the fluid dispensing system; and
   a second valve in fluid communication with the first chamber, the second valve suitable to control fluid communication between an air source and the first chamber.
26. The system of clause 22-25, further comprising a treatment chamber suitable to receive the device.
27. The system of clause 26, wherein the treatment chamber further comprises:
   a first door suitable to receive the device, the first door securable when the device is in a first state; and
   a second door suitable to receive the device, the second door securable wherein the device is a second state.
28. The system of clauses 26-27, further comprising a gas dryer disposed within the treatment chamber, the gas dryer suitable to remove liquid from an exterior surface of the device utilizing compressed gas.
29. The system of clauses 22-28, further comprising at least two liquid sources in fluid communication with the fluid dispensing system.
30. The system of clauses 22-29, further comprising a second fluid dispensing system suitable to provide liquid and gas to a second lumen of the device.
31. The system of clauses 22-30, wherein the device is an endoscope.
32. A treatment system comprising:
   a treatment chamber suitable to receive a device comprising a lumen;
   a fluid connector suitable to provide fluid communication between a fluid dispensing system and the lumen;
   a fluid dispensing system suitable to provide liquid and gas to the lumen in a first stage and a second stage, the first stage suitable to pass fluid substantially comprised of liquid through the lumen and the second stage suitable to pass fluid substantially comprised of gas through the lumen, and the second stage is suitable to facilitate movement of the first stage;
   a first chamber comprising a cavity substantially comprised of gas, the first chamber in fluid communication with the fluid dispensing system;
   a first valve in fluid communication with the fluid dispensing system, the first valve suitable to control fluid communication between the first chamber and the fluid dispensing system; and
   a second valve in fluid communication with the first chamber, the second valve suitable to control fluid communication between an air source and the first chamber.
33. A method for treatment of a device comprising a lumen, the method comprising:
   connecting the lumen of the device in fluid communication with a fluid dispensing system of a treatment system; and
   passing liquid and gas through the lumen utilizing the fluid dispensing system, comprising
      subjecting the lumen to a first stage comprising passing fluid substantially comprised of liquid through the lumen, and
      subjecting the lumen to a second stage comprising passing fluid substantially comprised of a gas through the lumen, wherein the second stage facilitates the movement of the first stage.
34. The method of clause 33, further comprising monitoring a pressure of gas entering the fluid dispensing system while passing liquid and gas through the lumen.
35. The method of clauses 33-34, wherein the liquid is a first liquid comprising a soap solution, the method further comprising:
   passing a disinfectant and gas through the lumen utilizing the fluid dispensing system; and
   passing an alcohol and gas through the lumen utilizing the fluid dispensing system.
36. The method of clauses 33-35, further comprising substantially removing liquid from an exterior of the device utilizing compressed gas.
37. The method of clauses 33-36, further comprising:
   connecting a first chamber substantially comprising gas in fluid communication with a second chamber substantially comprising fluid, the fluid dispensing system comprising the second chamber; and
   connecting the second chamber in fluid communication with the lumen.
38. The method of clauses 33-37, further comprising cleaning an exterior of the device with a liquid stream.
39. The method of clauses 33-38, further comprising passing liquid and gas through a second lumen of the device utilizing a second fluid dispensing system.
40. The method of clauses 33-39, further comprising providing the device in a first state to a treatment chamber of the treatment system through a first door of the treatment chamber and removing the device in a second state from the treatment chamber through a second door of the treatment chamber.
41. The method of clauses 33-40, wherein the device is an endoscope.
42. A fluid dispensing system comprising:
   a fluid chamber comprising an inlet and an outlet, the inlet and outlet suitable to transport fluid;
   a diverter disposed within the fluid chamber and suitable to move within the fluid chamber, the diverter comprising:
      a first end suitable to limit transport of liquid through the inlet;
      a second end suitable to limit transport of liquid through the outlet; and
      a body extending from the first end to the second end, the body suitable to facilitate a relationship between the movement of the first end and the second end.
43. The system of clause 42, further comprising a fluid source in fluid communication with the inlet, wherein the inlet is suitable to transport liquid from the fluid source into the fluid chamber.
44. The system of clauses 42-43, wherein at least one of the first end and the second end comprise a seal.
45. The system of clauses 44, wherein the seal is an O-ring.
46. The system of clauses 42-45, further comprising:
   a gas port in fluid communication with the fluid chamber and comprising a valve, the valve comprising a first position suitable to transport gas through the gas port into the fluid chamber to generate a sealing pressure, and the valve comprising a second position suitable to limit transport of a gas through the gas port, wherein the diverter is suitable to prevent fluid transport through the inlet with the first end responsive to the sealing pressure.
47. The system of clause 46, further comprising a resilient member operatively coupled to the diverter, the resilient member suitable to move the diverter to enable transport of fluid through the inlet of the fluid chamber responsive to a filling pressure in the fluid chamber, wherein the filling pressure is less than the sealing pressure.
48. The system of clause 47, wherein the resilient member is suitable to move the diverter to limit transport of fluid through the outlet of the fluid chamber.
49. The system of clauses 46-48, wherein the sealing pressure is suitable to expel at least one of liquid and gas from the fluid chamber through the outlet.
50. The system of clauses 46-49, wherein the valve is a solenoid valve.
51. The system of clauses 46-50, wherein the inlet is substantially occluded responsive to the sealing pressure within the fluid chamber.
52. The system of clauses 46-51, further comprising:
   a compressed gas chamber comprising a gas inlet and a gas outlet;
   the gas inlet comprising a source valve, the source valve comprising a first position suitable to transport gas into the gas chamber and a second position suitable to limit transport of gas into the gas chamber; and
   the gas outlet in fluid communication with the gas port, the gas outlet comprising an outlet valve, the outlet valve comprising a first position suitable to transport gas into the gas port from the gas chamber and a second position suitable to limit transport of gas into the gas port from the gas chamber.
53. The system of clauses 46-52,
   wherein the diverter is configured to move from a first diverter position to a second diverter position responsive to the sealing pressure within the fluid chamber,
   the first position suitable to facilitate transport of liquid into the fluid chamber through the inlet and suitable to limit transport of liquid through the outlet, and
   the second position suitable to limit transport of liquid into the fluid chamber through the inlet and suitable to facilitate transport of liquid through the outlet.
54. The system of clauses 42-53, wherein the outlet is in fluid communication with a lumen of an endoscope.
55. The system of clauses 42-54, wherein the system is configurable to an orientation suitable to gravity fill the fluid chamber with liquid through the inlet.
56. A treatment system comprising:
   a pressure regulator;
   a first purge channel configured to be connected to a first endoscope lumen;
   a second purge channel configured to be connected to a second endoscope lumen;
   a first reservoir disposed between and connected to the pressure regulator and the first purge channel; and
   a second reservoir disposed between and connected to the pressure regulator and the second purge channel.
57. The treatment system of clause 56, further comprising a first valve disposed between the first reservoir and the first purge channel and a second valve disposed between the second reservoir and the second purge channel.
58. The treatment system of clause 57, wherein the first valve and the second valve each comprise a solenoid valve.
59. The treatment system of clause 58, further comprising a first pressure sensor connected to the first reservoir and a second pressure sensor connected to the second reservoir.
60. The treatment system of clause 59, further comprising a third valve disposed between and connected to the pressure regulator and the first reservoir.
61. The treatment system of clause 60, wherein the pressure regulator comprises a first pressure regulator, and wherein the third valve is further connected to a second pressure regulator.
62. The treatment system of clause 61, wherein the first pressure regulator is configured to output a pressure of about 30 psi.
63. The treatment system of clause 63, wherein the third valve is configured to place either the first pressure regulator or the second pressure regulator in fluid communication with the first reservoir.
64. The treatment system of clause 64, wherein the third valve is also connected to the second reservoir.
65. A method of using a treatment system to purge a cleaning liquid from a first lumen and a second lumen of an endoscope, the first lumen connected to a first line of the treatment system and the second lumen connected to a second line of the treatment system, the method comprising:
   charging a first reservoir on the first line with a gas;
   charging a second reservoir on the second line with the gas;
   flowing the gas from the first reservoir through the first lumen to purge the first lumen of the endoscope; and
   flowing the gas from the second reservoir through the second lumen to purge the second lumen of the endoscope.
66. The method of clause 65, wherein the steps of charging the first reservoir and the second reservoir occur simultaneously.
67. The method of clause 65, wherein the step of charging the first reservoir includes pressurizing the first reservoir to a pressure between about 25 psi and 35 psi.
68. The method of clause 67, wherein the step of charging the first reservoir includes pressurizing the first reservoir to a pressure of about 30 psi.
69. The method of clause 67, wherein the steps of charging the first reservoir and the second reservoir each comprise activating a pressure regulator.
70. The method of clause 67, further comprising checking the first line and the second line for a blockage.
71. The method of clause 67, further comprising repeating the steps of charging the first reservoir, charging the second reservoir, flowing the gas from the first reservoir through the first lumen, and flowing the gas from the second reservoir through the second lumen.

In summary, numerous benefits have been described which result from employing the concepts described herein. The foregoing description of the one or more examples has been presented for purposes of illustration and description. It is not intended to be exhaustive or limiting to the precise form disclosed. Modifications or variations are possible in light of the above teachings. The one or more examples were chosen and described in order to illustrate principles and practical application to thereby enable one of ordinary skill in the art to utilize the various embodiments and with various modifications as are suited to the particular use contemplated. It is intended that the claims submitted herewith define the overall scope.

While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed, and not as more narrowly defined by particular illustrative aspects provided herein

What is claimed is:

1. A fluid dispensing system comprising:
a fluid chamber comprising an inlet and an outlet, the inlet and outlet suitable to transport fluid;
the inlet comprising a body and a gate, the body comprising an opening suitable to transport liquid into the fluid chamber, the gate suitable to substantially occlude the opening to limit the transport of liquid through the opening responsive to a sealing pressure within the fluid chamber;
a gas port in fluid communication with the fluid chamber and comprising a valve, the valve comprising a first position suitable to transport gas through the gas port into the fluid chamber to generate the sealing pressure, and a second position suitable to limit transport of gas through the gas port;
the outlet is configured to generate a backpressure to limit transport of liquid through the outlet responsive to a filling pressure in the fluid chamber, the filling pressure is less than the sealing pressure; and
the sealing pressure is suitable to expel at least one of liquid and gas from the fluid chamber through the outlet.

2. A fluid dispensing method comprising:
filling a portion of a fluid chamber with liquid by providing liquid through an inlet of the fluid chamber;
generating a sealing pressure with gas in the fluid chamber and limiting additional filling of the fluid chamber through the inlet; and
dispensing liquid from the fluid chamber responsive to the sealing pressure, the dispensing comprises expelling liquid through an outlet of the fluid chamber utilizing gas.

3. The method of claim 2, further comprising generating backpressure in the outlet of the fluid chamber in order to facilitate filling of the fluid chamber.

4. The method of claim 3, wherein the filling of the portion of the fluid chamber with liquid further comprises gravity filling.

5. The method of claim 4, wherein generating the sealing pressure further comprises activating a solenoid valve to provide gas to the fluid chamber.

6. The method of claim 5, wherein the inlet comprises a gate and limiting additional filling of the fluid chamber further comprises changing the configuration of the gate utilizing the sealing pressure.

7. The method of claim 6, further comprising expelling gas through an outlet of the fluid chamber.

8. The method of claim 7, wherein dispensing liquid from the fluid chamber further comprises passing liquid through a lumen of an endoscope.

9. A treatment system comprising the fluid dispensing system of any one of claim 8.

10. A treatment system comprising:
a treatment chamber suitable to receive a device comprising a lumen;
a fluid connector suitable to provide fluid communication between a fluid dispensing system and the lumen;
a fluid dispensing system suitable to provide liquid and gas to the lumen in a first stage and a second stage, the first stage suitable to pass fluid substantially comprised of liquid through the lumen and the second stage suitable to pass fluid substantially comprised of gas through the lumen, and the second stage is suitable to facilitate movement of the first stage;
a first chamber comprising a cavity substantially comprised of gas, the first chamber in fluid communication with the fluid dispensing system;
a first valve in fluid communication with the fluid dispensing system, the first valve suitable to control fluid communication between the first chamber and the fluid dispensing system; and
a second valve in fluid communication with the first chamber, the second valve suitable to control fluid communication between an air source and the first chamber.

11. A method for treatment of a device comprising a lumen, the method comprising:
connecting the lumen of the device in fluid communication with a fluid dispensing system of a treatment system; and
passing liquid and gas through the lumen utilizing the fluid dispensing system, comprising
subjecting the lumen to a first stage comprising passing fluid substantially comprised of liquid through the lumen, and
subjecting the lumen to a second stage comprising passing fluid substantially comprised of a gas through the lumen, wherein the second stage facilitates the movement of the first stage.

12. The method of claim 11, further comprising monitoring a pressure of gas entering the fluid dispensing system while passing liquid and gas through the lumen.

13. The method of claim 12, wherein the liquid is a first liquid comprising a soap solution, the method further comprising:
passing a disinfectant and gas through the lumen utilizing the fluid dispensing system; and
passing an alcohol and gas through the lumen utilizing the fluid dispensing system.

14. The method of claim 13, further comprising substantially removing liquid from an exterior of the device utilizing compressed gas.

15. The method of claim 14, further comprising:
connecting a first chamber substantially comprising gas in fluid communication with a second chamber substantially comprising fluid, the fluid dispensing system comprising the second chamber; and
connecting the second chamber in fluid communication with the lumen.

16. The method of claim 15, further comprising cleaning an exterior of the device with a liquid stream.

17. The method of claim 16, further comprising passing liquid and gas through a second lumen of the device utilizing a second fluid dispensing system.

18. The method of claim 17, further comprising providing the device in a first state to a treatment chamber of the treatment system through a first door of the treatment chamber and removing the device in a second state from the treatment chamber through a second door of the treatment chamber.

19. The method of claim 18, wherein the device is an endoscope.

20. A fluid dispensing system comprising:
a fluid chamber comprising an inlet and an outlet, the inlet and outlet suitable to transport fluid;
a diverter disposed within the fluid chamber and suitable to move within the fluid chamber, the diverter comprising:
a first end suitable to limit transport of liquid through the inlet;
a second end suitable to limit transport of liquid through the outlet; and
a body extending from the first end to the second end, the body suitable to facilitate a relationship between the movement of the first end and the second end.

21. The system of claim 20, further comprising a fluid source in fluid communication with the inlet, wherein the inlet is suitable to transport liquid from the fluid source into the fluid chamber.

22. The system of claim 21, wherein at least one of the first end and the second end comprise a seal.

23. The system of claim 22, wherein the seal is an O-ring.

24. The system of claim 23, further comprising:
a gas port in fluid communication with the fluid chamber and comprising a valve, the valve comprising a first position suitable to transport gas through the gas port into the fluid chamber to generate a sealing pressure, and the valve comprising a second position suitable to limit transport of a gas through the gas port, wherein the diverter is suitable to prevent fluid transport through the inlet with the first end responsive to the sealing pressure.

25. The system of claim 24, further comprising a resilient member operatively coupled to the diverter, the resilient member suitable to move the diverter to enable transport of fluid through the inlet of the fluid chamber responsive to a filling pressure in the fluid chamber, wherein the filling pressure is less than the sealing pressure.

26. The system of claim 25, wherein the resilient member is suitable to move the diverter to limit transport of fluid through the outlet of the fluid chamber.

27. The system of claim 26, wherein the sealing pressure is suitable to expel at least one of liquid and gas from the fluid chamber through the outlet.

28. The system of claim 27, wherein the valve is a solenoid valve.

29. The system of claim 28, wherein the inlet is substantially occluded responsive to the sealing pressure within the fluid chamber.

30. The system of claim 29, further comprising:
a compressed gas chamber comprising a gas inlet and a gas outlet;
the gas inlet comprising a source valve, the source valve comprising a first position suitable to transport gas into the gas chamber and a second position suitable to limit transport of gas into the gas chamber; and
the gas outlet in fluid communication with the gas port, the gas outlet comprising an outlet valve, the outlet valve comprising a first position suitable to transport gas into the gas port from the gas chamber and a second position suitable to limit transport of gas into the gas port from the gas chamber.

31. The system of claim 30,
wherein the diverter is configured to move from a first diverter position to a second diverter position responsive to the sealing pressure within the fluid chamber,
the first position suitable to facilitate transport of liquid into the fluid chamber through the inlet and suitable to limit transport of liquid through the outlet, and
the second position suitable to limit transport of liquid into the fluid chamber through the inlet and suitable to facilitate transport of liquid through the outlet.

32. The system of claim 31, wherein the outlet is in fluid communication with a lumen of an endoscope.

33. The system of claim 32, wherein the system is configurable to an orientation suitable to gravity fill the fluid chamber with liquid through the inlet.

34. A treatment system comprising:
a pressure regulator;
a first purge channel configured to be connected to a first endoscope lumen;
a second purge channel configured to be connected to a second endoscope lumen;
a first reservoir disposed between and connected to the pressure regulator and the first purge channel; and
a second reservoir disposed between and connected to the pressure regulator and the second purge channel.

35. The treatment system of claim 34, further comprising a first valve disposed between the first reservoir and the first purge channel and a second valve disposed between the second reservoir and the second purge channel.

36. The treatment system of claim 35, wherein the first valve and the second valve each comprise a solenoid valve.

37. The treatment system of claim 36, further comprising a first pressure sensor connected to the first reservoir and a second pressure sensor connected to the second reservoir.

38. The treatment system of claim 37, further comprising a third valve disposed between and connected to the pressure regulator and the first reservoir.

39. The treatment system of claim 38, wherein the pressure regulator comprises a first pressure regulator, and wherein the third valve is further connected to a second pressure regulator.

40. The treatment system of claim 39, wherein the first pressure regulator is configured to output a pressure of about 30 psi.

41. The treatment system of claim 40, wherein the third valve is configured to place either the first pressure regulator or the second pressure regulator in fluid communication with the first reservoir.

42. The treatment system of claim 41, wherein the third valve is also connected to the second reservoir.

43. A method of using a treatment system to purge a cleaning liquid from a first lumen and a second lumen of an endoscope, the first lumen connected to a first line of the treatment system and the second lumen connected to a second line of the treatment system, the method comprising:
charging a first reservoir on the first line with a gas;
charging a second reservoir on the second line with the gas;
flowing the gas from the first reservoir through the first lumen to purge the first lumen of the endoscope; and
flowing the gas from the second reservoir through the second lumen to purge the second lumen of the endoscope.

44. The method of claim 43, wherein the steps of charging the first reservoir and the second reservoir occur simultaneously.

45. The method of claim 44, wherein the step of charging the first reservoir includes pressurizing the first reservoir to a pressure between about 25 psi and 35 psi.

46. The method of claim 45, wherein the step of charging the first reservoir includes pressurizing the first reservoir to a pressure of about 30 psi.

47. The method of claim 45, wherein the steps of charging the first reservoir and the second reservoir each comprise activating a pressure regulator.

48. The method of claim 45, further comprising checking the first line and the second line for a blockage.

49. The method of claim 45, further comprising repeating the steps of charging the first reservoir, charging the second reservoir, flowing the gas from the first reservoir through the first lumen, and flowing the gas from the second reservoir through the second lumen.
